(19) United States
(12) Plant Patent Application Publication
(10) Pub. No.: US 2014/0020146 P1
(43) Pub. Date: Jan. 16, 2014

(54) Osteospermum plant named 'SAKOST8436'

(50) ;

(71) Applicant: Niels G. Kristensen, Grauballe (DK)

(72) Inventor: Niels G. Kristensen, Grauballe (DK)

(73) Assignee: Sakata Ornamentals Europe A/S, Marslev (DK)

(21) Appl. No.: 13/507,573

(22) Filed: Jul. 11, 2012

(51) Int. Cl. A01H 5/00

(52) U.S. Cl. PLT/360

(57) ABSTRACT

A new Osteospermum plant particularly distinguished by having young flowers with almost white ray florets and white-grey disc florets and mature flowers with purple stripes on ray florets and a purple colored ring around the disc (at base of ray florets) and a semi-erect plant habit is disclosed.

### GENUS AND SPECIES

Osteospermum hybrida

### VARIETY DENOMINATION

‘SAKOST8436’

### BACKGROUND OF THE NEW PLANT

The present invention comprises a new and distinct variety of Osteospermum, botanically known as Osteospermum hybrida, and hereinafter referred to by the variety name ‘SAKOST8436’. ‘SAKOST8436’ originated from a hybridization of Osteospermum proprietary breeding line ‘206144’ and Osteospermum proprietary breeding line ‘206145’ in Marslev, Denmark. The female parent, ‘206144’ has a white to very light purple flower color and white-grey disc florets. The male parent, ‘206145’ has a purple flower color with yellow disc florets.

From May 2007 to June 2007, the two Osteospermum parental lines ‘206144’ and ‘206145’ were crossed and seeds were obtained. The seeds were sown and 102 plants were grown in pots for evaluation. Out of 102 F₁ lines, plant number 36 was selected which has young flowers with white ray florets and white-grey disc florets and mature flowers with purple stripes on ray florets and a purple colored ring around the disc (at base of ray florets).

In June 2008, plant number 36 was vegetatively propagated by cuttings and re-evaluated in an open field and a greenhouse. Plant number 36 was given the breeder code number ‘208090’ and the stability of the distinct characteristics of this variety was confirmed.

In June 2009, plants were evaluated again in pots and in an open field. The selection was named ‘SAKOST8436’ and was found to reproduce true to type in successive generations of asexual propagation in Salinas, Calif.

### SUMMARY

The following are the most outstanding and distinguishing characteristics of this new variety when grown under normal horticultural practices in Salinas, Calif.

1. Young flowers with almost white ray florets and white-grey disc florets;

2. Mature flowers with purple stripes on ray florets and a purple colored ring around the disc (at base of ray florets); and

3. Semi-erect plant habit.

### DESCRIPTION OF THE PHOTOGRAPHS

This new Osteospermum plant is illustrated by the accompanying photographs which show the plant's overall plant habit including form, foliage, and flowers. The photographs are of a plant grown four months from transplant (five months from sticking) into 4-inch pots from rooted cuttings in Salinas, Calif. under greenhouse conditions in the spring of 2012. The colors shown are as true as can be reasonably obtained by conventional photographic procedures.

FIG. 1 shows the overall plant habit of the plant grown in a pot.

FIG. 2 shows the mature inflorescence of the plant.

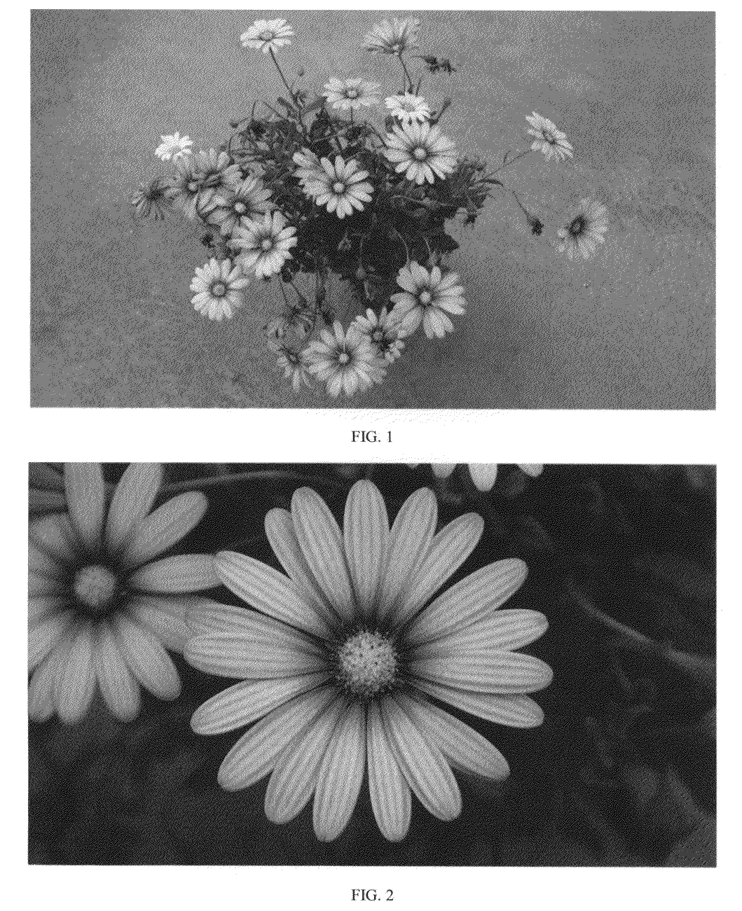

### DESCRIPTION OF THE NEW VARIETY

The following detailed descriptions set forth the distinctive characteristics of ‘SAKOST8436’. The data which define these characteristics were collected from asexual reproductions carried out in Salinas, Calif. Data was collected on plants grown approximately four months from transplant (five months from sticking) into 4-inch pots under greenhouse conditions in Salinas, Calif. in the spring of 2012. Color references are to The R.H.S. Colour Chart of The Royal Horticultural Society of London (R.H.S.), 4^{th} edition.
Classification:
   Family.—Compositae.
   Botanical.—Osteospermum hybrida.
   Common.—Osteospermum, Cape Daisy, South African Daisy.
   Designation.—‘SAKOST8436’.
Parentage:
   Female parent.—The proprietary Osteospermum line ‘206144’ (unpatented).
   Male parent.—The proprietary Osteospermum line ‘206145’ (unpatented).
Growth:
   Time to produce a rooted cutting.—Cuttings will colonize a 2.5 cm diameter by 2.5 cm tall greenhouse tray cell with peat-based plant media in approximately four weeks. Cuttings are dipped in a normal dilution (1:9) of Dip ‘N Grow root inducing solution in water. The trays are misted hourly during rooting.
   Environmental conditions for plant growth.—Rooted cuttings are transplanted to pots with a 16 cm diameter, one plant per pot. Peat-based growing media is used. The pots are watered using a 150 ppm to 200 ppm fertilizer solution using 18-8-18 fertilizer. The soil is allowed to dry between watering. During the first few weeks after transplanting, the plants should have evening temperatures around 15° C. to 18° C. for good root growth. When plants reach 7.5 cm to 10 cm in height they are pinched back to 5 to 6 leaves to promote branching. Spring and summer daytime high temperatures in Salinas, Calif. where the data was collected, range from 16° C. to 25° C.
   Time to bloom from propagation.—Approximately four weeks when rooted vegetative cuttings are transferred to a 5-inch diameter pot. Flowering season is all year in the United States. Vernalization is not required to induce flowering.
Plant description:
   Habit.—Semi-erect.
   Life cycle.—Perennial.
   Form.—Semi-erect.
   Height (from soil line to first node).—2.0 cm.
   Height (from soil line to top of foliage).—32.0 cm to 34.0 cm.
   Width.—44.0 cm to 45.0 cm.
Stems:
   General.—Multiple; circular in cross-section.
   Stem length (total).—23.0 cm to 24.0 cm.
   Diameter.—0.2 cm.
   Internode length.—2.0 cm.
   Color.—RHS 143B (Green) with RHS 187A (Greyed-purple) anthocyanin.
   Pubescence.—Slight. Color: RHS N155A (White) Shape: Short, hair-like fibers.
   Anthocyanin color.—Slight; RHS 187A (Greyed-purple).
Branches:
   General.—Circular in cross-section.
   Number of main branches.—One.
   Total number of branches.—Eight.
   Length.—14.0 cm to 15.0 cm, approximately 1.0 cm from soil line to first node, 2.5 cm between first and second nodes.
   Diameter.—0.5 cm.
   Internode length.—2.5 cm.
   Color.—RHS 143B (Green) with slight anthocyanin.
   Pubescence.—Slight.
   Pubescence shape.—Short, hair-like fibers.
   Pubescence color.—RHS N155A (White).
   Anthocyanin color.—RHS 187A (Greyed-purple).
Leaves:
   Arrangement.—Alternate.
   Shape.—Oblanceolate.
   Apex.—Acute.
   Base.—Attenuate.
   Margin.—Serrate.
   Venation.—Pinnate.
   Surface (both surfaces).—Dull.
   Surface pubescence (both surfaces).—Slight, mainly around edge.
   Surface pubescence color.—RHS N155A (White).
   Attachment.—Decurrant.
   Length.—7.0 cm.
   Width.—2.5 cm.
   Thickness.—Less than 0.1 cm.
   Color.—Upper surface: RHS 137A (Green) Lower surface: RHS 137C (Green).
   Venation color.—Upper surface: RHS 144A (Yellow-green) Lower surface: RHS 144A (Yellow-green).
Inflorescence:
   Number per plant.—22.
   Type.—Capitulum (head); disc florets are perfect and ray florets are carpellate.
   Flowering habit.—Determinate.
   Lastingness of inflorescence on plant.—3 to 4 days.
   Fragrance.—Absent.
   Seed production.—None observed.
   Diameter.—7.0 cm to 8.0 cm.
   Depth.—2.0 cm.
   Petal (ray floret) number.—19 per inflorescence.
   Disc diameter.—1.3 cm.
   Disc floret number.—60 to 80 per inflorescence.
   Peduncle length.—10.0 cm from inflorescence to first node.
   Peduncle diameter.—0.2 cm.
   Peduncle color.—RHS 144A (Yellow-green).
   Peduncle texture.—Dull; slight pubescence.
   Phyllaries.—Arrangement: 19 per inflorescence; free; arranged symmetrically Length: 1.0 cm Width: 0.1 cm Apex: Acute Margin: Entire Shape: Linear; acute Color: Upper surface: RHS 144B (Yellow-green) Lower surface: RHS 144A (Yellow-green).
Ray florets (ligules):
   Corolla.—One petal per ray floret; only the outer row of florets are the ray florets.
   Number of ray florets.—19 per inflorescence.
   Length.—3.5 cm.
   Width.—0.9 cm.
   Shape.—Lanceolate.
   Apex.—Acute.
   Margin.—Entire.
   Color.—Upper surface: RHS N155A (White) with RHS 77A (Purple) stripes (from base to tip) and RHS 77A (Purple) at base Lower surface: RHS 75B (Purple) with RHS 79B (Purple) stripes from base to tip and RHS 79B (Purple) at base.
   Pubescence.—Glabrous.
Disc florets:
   Color.—RHS N88A (Violet).
   Shape.—Tubular.
   Apex.—Pointed.
   Texture.—Dull, pubescent.
   Size.—Length: 0.5 cm Width: 0.1 cm.
Reproductive organs:
   Ovary.—Superior.
   Pistil form.—One style with two stigma branches.
   Pistil length.—0.5 cm.
   Stigma color.—RHS 155A (White).
   Style color.—RHS 187B (Greyed-purple).
   Stamens.—5; fused into a single tube.
   Anther color.—RHS 187A (Greyed-purple).
   Pollen color.—RHS 17A (Yellow-orange).
   Filament color.—RHS 155A (White).
Disease and insect resistance: No known resistance or susceptibility

### COMPARISON WITH PARENTAL LINES AND KNOWN VARIETY

‘SAKOST8436’ is a distinct variety of Osteospermum is a distinct variety of Osteospermum owing to its young flowers with almost white ray florets and white-grey disc florets and mature flowers with purple stripes on ray florets and a purple colored ring around the disc (at base of ray florets). ‘SAKOST8436’ is distinguished from its parent as described in Table 1 (color references are to The Royal Horticultural Society Colour Chart, 4^{th} edition):

TABLE 1
Comparison with Parental Lines

| | | Male Parent | Female Parent |
|---|---|---|---|
| Characteristic | ‘SAKOST8436’ | ‘206145’ | ‘206144’ |
| Ray floret | RHS N155A (White) | Purple | White to very |
| (ligule) color, | with RHS 77A (Purple) | | light purple |
| upper surface: | stripes (from base to | | |
| | tip) and RHS 77A | | |
| | (Purple) at base | | |
| Disc floret color | RHS N88A (Violet) | Yellow | White-grey |

‘SAKOST8436’ is a distinct variety of Osteospermum owing to its young flowers with almost white ray florets and white-grey disc florets and mature flowers with purple stripes on ray florets and a purple colored ring around the disc (at base of ray florets). ‘SAKOST8436’ is most similar to the Osteospermum plant named ‘SAKOST8181’ (U.S. Plant Pat. No. 23,502). Differences between the two varieties are described in Table 2 (color references are to The Royal Horticultural Society Colour Chart, 4^{th} edition):

TABLE 2
Comparison with Similar Variety

| Characteristic | ‘SAKOST8436’ | ‘SAKOST8181’ |
|---|---|---|
| Ray floret (ligule) | RHS N155A (White) with RHS | RHS 77A (Purple) |
| color, upper surface: | 77A (Purple) stripes (from base | |
| | to tip) and RHS 77A (Purple) at | |
| | base | |
| Ray floret (ligule) | RHS 75B (Purple) with RHS | RHS 77B (Purple) |
| color, lower surface: | 79B (Purple) stripes from base | |
| | to tip and RHS 79B (Purple) at | |
| | base | |

I claim:

1. A new and distinct variety of Osteospermum plant as shown and described herein.
